(19) United States
(12) Patent Application Publication
(10) Pub. No.: US 2015/0335688 A1
(43) Pub. Date: Nov. 26, 2015

(54) PROBIOTIC COMBINATIONS AND USES THEREOF

(71) Applicant: Medlab IP Pty Ltd, Alexandria (AU)

(72) Inventors: Samantha COULSON, Engadine (AU); Sean HALL, Sydney (AU); Luis VITETTA, Varsity Lakes (AU)

(21) Appl. No.: 14/718,636

(22) Filed: May 21, 2015

(30) Foreign Application Priority Data
May 21, 2014 (AU) 2014901889

(51) Int. Cl. A61K 35/747; A61K 31/122; A61K 31/205; A61K 31/505; A61K 35/745; A61K 35/744

(52) U.S. Cl. CPC A61K 35/747; A61K 35/745; A61K 35/744; A61K 31/205; A61K 31/505; A61K 31/122; A61K 2035/115

(57) ABSTRACT

The present disclosure relates generally to compositions and methods to provide probiotic supplements useful for the therapeutic and/or prophylactic treatment, amelioration and/or regulation of disease states or pathological conditions. More specifically the present disclosure relates to compositions comprising at least one probiotic microorganism, optionally in combination with one or more of orotic acid or a salt thereof, carnitine and/or coenzyme Q10, and to uses of such compositions for the treatment or prevention of fatigue, for promoting synthesis of adenosine triphosphate (ATP), for muscle repair and the treatment of muscle degeneration, muscle aches and inflammation, and/or for boosting energy levels, by improving mitochondrial function.

### FIELD OF THE ART

The present disclosure relates generally to compositions and methods to provide probiotic supplements useful for the therapeutic and/or prophylactic treatment, amelioration and/or regulation of disease states or pathological conditions. More specifically the present disclosure relates to compositions comprising at least one probiotic microorganism, optionally in combination with one or more of orotic acid or a salt thereof, carnitine and/or coenzyme Q10, and to uses of such compositions for the treatment or prevention of fatigue, for promoting synthesis of adenosine triphosphate (ATP), for muscle repair and the treatment of muscle degeneration, muscle aches and inflammation, and/or for boosting energy levels, by improving mitochondrial function.

### BACKGROUND

Health problems such as fatigue, energy depletion, lack of concentration, demotivation and sore muscles can result from stress, sleep deprivation, a diet high in processed foods, age, and the “non-stop” culture of modern society which often prevents individuals from taking adequate rest. Such health problems can cause discomfort and decrease the quality of life of an individual, as well as decreasing their efficiency to work.

There is a need for the development of improved compositions and formulations to assist individuals with the treatment and prevention of fatigue, energy depletion, lack of concentration, demotivation and sore muscles and associated symptoms thereof.

Skeletal muscle experiences a decline in regenerative potential with ageing, which is manifested by decreased generation of myogenic fibers and their replacement with fibrous tissue in humans. Diminished muscle regeneration is most apparent after injury, with a protracted and incomplete recovery in aged humans. Muscle degeneration can also result from infection, disease or trauma. Progressive muscle degeneration and wasting are also characteristic of muscular dystrophies. Regardless of the cause, there is a need for the development of effective treatments to combat muscle degeneration and associated difficulties such as muscle aches and inflammation.

### SUMMARY OF THE DISCLOSURE

Provided herein are probiotic compositions comprising bacterial strains selected from Lactobacillus rhamnosus, Lactobacillus plantarum, Lactobacillus bulgaricus, Lactobacillus gasseri, Lactobacillus reuteri, Lactobacillus paracasei, Lactobacillus casei, Lactobacillus acidophilus, Lactobacillus fermentum, Lactobacillus salvarius, Lactococcus lactis, Streptococcus thermophilus, Bifidobacterium breve, Bifidobacterium bifidum, Bifidobacterium animalis subsp. lactis, Bifidobacterium animalis subsp. animalis, Bifidobacterium infantis, Bifidobacterium longum and Bifidobacterium pseudocatenulatum, and uses of such compositions for therapeutic or prophylactic treatment, amelioration and/or regulation of a disease state in a subject.

In a first aspect the present disclosure provides a method for the treatment or prevention of fatigue, the method comprising administering to a subject in need thereof an effective amount of one or more probiotic bacterial strains selected from Lactobacillus rhamnosus, Lactobacillus plantarum, Lactobacillus bulgaricus, Lactobacillus gasseri, Lactobacillus reuteri, Lactobacillus paracasei, Lactobacillus casei, Lactobacillus acidophilus, Lactobacillus fermentum, Lactobacillus salvarius, Lactococcus lactis Streptococcus thermophilus, Bifidobacterium breve, Bifidobacterium bifidum, Bifidobacterium animalis subsp. lactis (B. lactis), Bifidobacterium animalis subsp. animalis (B. animalis), Bifidobacterium infantis, Bifidobacterium longum and Bifidobacterium pseudocatenulatum.

The fatigue may result from, or be associated with, stress, sleep deprivation, diet, age, and/or sore muscles.

The treatment may further comprise administering to the subject an effective amount of one or more of the following: orotic acid or a salt thereof; carnitine; and coenzyme Q10.

The orotic acid may be present as an orotate salt. The orotate may be selected from, for example, magnesium orotate, zinc orotate, calcium orotate, chromium orotate, potassium orotate, copper orotate, iron orotate, manganese orotate, sodium orotate, choline orotate and lithium orotate. In an exemplary embodiment the orotate is magnesium orotate.

Typically the carnitine is L-carnitine, more typically acetyl-L-carnitine.

The coenzyme Q10 may be in a reduced form, for example as ubiquinol.

The one or more probiotic bacterial strains may comprise at least one Lactobacillus species, at least one Bifidobacterium species and/or at least one Streptococcus species. The Lactobacillus may be L. rhamnosus or L. acidophilus. The L. rhamnosus may be L. rhamnosus GG. The Bifidobacterium may be B. bifidum. The Streptococcus may be S. thermophilus.

The method may comprise the administration of a multi-strain combination of probiotic organisms. In one exemplary embodiment, the combination comprises L. rhamnosus, B. bifidum, and S. thermophilus. In a further exemplary embodiment the combination comprises L. acidophilus, B. bifidum, and S. thermophilus. In accordance with these exemplary embodiments, the combination may further comprise magnesium orotate, coenzyme Q10 (for example as ubiquinol), and optionally L-carnitine (for example as acetyl-L-carnitine).

In one embodiment the combination comprises or consists essentially of L. acidophilus, B. bifidum, S. thermophilus, magnesium orotate and coenzyme Q10.

The method may further comprise administration to the subject of one or more prebiotic components. The prebiotic may be administered in the same composition as the probiotic strain(s) or in a different composition.

In a second aspect the present disclosure provides a probiotic composition for the treatment or prevention of fatigue, wherein the composition comprises two or more bacterial strains selected from Lactobacillus rhamnosus, Lactobacillus plantarum, Lactobacillus bulgaricus, Lactobacillus gasseri, Lactobacillus reuteri, Lactobacillus paracasei, Lactobacillus casei, Lactobacillus acidophilus, Lactobacillus fermentum, Lactobacillus salvarius, Lactococcus lactis Streptococcus thermophilus, Bifidobacterium breve, Bifidobacterium bifidum, Bifidobacterium animalis subsp. lactis (B. lactis), Bifidobacterium animalis subsp. animalis (B. animalis), Bifidobacterium infantis, Bifidobacterium longum and Bifidobacterium pseudocatenulatum.

The fatigue may result from, or be associated with, stress, sleep deprivation, diet, age, and/or sore muscles.

The composition may further one or more of the following: orotic acid or a salt thereof; carnitine; and coenzyme Q10.

The orotic acid may be present as an orotate salt. The orotate may be selected from, for example, magnesium orotate, zinc orotate, calcium orotate, chromium orotate, potassium orotate, copper orotate, iron orotate, manganese orotate, sodium orotate, choline orotate and lithium orotate. In an exemplary embodiment the orotate is magnesium orotate.

Typically the carnitine is L-carnitine, more typically acetyl-L-carnitine.

The coenzyme Q10 may be in a reduced form, for example as ubiquinol.

The two or more probiotic bacterial strains may comprise at least one Lactobacillus species, at least one Bifidobacterium species and/or at least one Streptococcus species. The Lactobacillus may be L. rhamnosus or L. acidophilus. The L. rhamnosus may be L. rhamnosus GG. The Bifidobacterium may be B. bifidum. The Streptococcus may be S. thermophilus.

The composition may comprise a multi-strain combination of probiotic organisms. In one exemplary embodiment, the combination comprises L. rhamnosus, B. bifidum, and S. thermophilus. In a further exemplary embodiment the combination comprises L. acidophilus, B. bifidum, and S. thermophilus. In accordance with these exemplary embodiments, the combination may further comprise magnesium orotate, coenzyme Q10 (for example as ubiquinol), and optionally L-carnitine (for example as acetyl-L-carnitine).

In one embodiment the composition comprises or consists essentially of L. acidophilus, B. bifidum, S. thermophilus, magnesium orotate and coenzyme Q10.

The composition may further comprise one or more prebiotic components.

In a third aspect the present disclosure provides a method for promoting synthesis of adenosine triphosphate (ATP), the method comprising administering to a subject in need thereof an effective amount of one or more probiotic bacterial strains selected from Lactobacillus rhamnosus, Lactobacillus plantarum, Lactobacillus bulgaricus, Lactobacillus gasseri, Lactobacillus reuteri, Lactobacillus paracasei, Lactobacillus casei, Lactobacillus acidophilus, Lactobacillus fermentum, Lactobacillus salvarius, Lactococcus lactis Streptococcus thermophilus, Bifidobacterium breve, Bifidobacterium bifidum, Bifidobacterium animalis subsp. lactis (B. lactis), Bifidobacterium animalis subsp. animalis (B. animalis), Bifidobacterium infantis, Bifidobacterium longum and Bifidobacterium pseudocatenulatum.

The subject may suffer fatigue resulting from, or associated with, for example, stress, sleep deprivation, diet, age, and/or sore muscles.

The treatment may further comprise administering to the subject an effective amount of one or more of the following: orotic acid or a salt thereof; carnitine; and coenzyme Q10.

The orotic acid may be present as an orotate salt. The orotate may be selected from, for example, magnesium orotate, zinc orotate, calcium orotate, chromium orotate, potassium orotate, copper orotate, iron orotate, manganese orotate, sodium orotate, choline orotate and lithium orotate. In an exemplary embodiment the orotate is magnesium orotate.

Typically the carnitine is L-carnitine, more typically acetyl-L-carnitine.

The coenzyme Q10 may be in a reduced form, for example as ubiquinol.

The one or more probiotic bacterial strains may comprise at least one Lactobacillus species, at least one Bifidobacterium species and/or at least one Streptococcus species. The Lactobacillus may be L. rhamnosus or L. acidophilus. The L. rhamnosus may be L. rhamnosus GG. The Bifidobacterium may be B. bifidum. The Streptococcus may be S. thermophilus.

The method may comprise the administration of a multi-strain combination of probiotic organisms. In one exemplary embodiment, the combination comprises L. rhamnosus, B. bifidum, and S. thermophilus. In a further exemplary embodiment the combination comprises L. acidophilus, B. bifidum, and S. thermophilus. In accordance with these exemplary embodiments, the combination may further comprise magnesium orotate, coenzyme Q10 (for example as ubiquinol), and optionally L-carnitine (for example as acetyl-L-carnitine).

In one embodiment the combination comprises or consists essentially of L. acidophilus, B. bifidum, S. thermophilus, magnesium orotate and coenzyme Q10.

The method may further comprise administration to the subject of one or more prebiotic components. The prebiotic may be administered in the same composition as the probiotic strain(s) or in a different composition.

In a fourth aspect the present disclosure provides a probiotic composition for the promoting synthesis of adenosine triphosphate (ATP), wherein the composition comprises two or more bacterial strains selected from Lactobacillus rhamnosus, Lactobacillus plantarum, Lactobacillus bulgaricus, Lactobacillus gasseri, Lactobacillus reuteri, Lactobacillus paracasei, Lactobacillus casei, Lactobacillus acidophilus, Lactobacillus fermentum, Lactobacillus salvarius, Lactococcus lactis Streptococcus thermophilus, Bifidobacterium breve, Bifidobacterium bifidum, Bifidobacterium animalis subsp. lactis (B. lactis), Bifidobacterium animalis subsp. animalis (B. animalis), Bifidobacterium infantis, Bifidobacterium longum and Bifidobacterium pseudocatenulatum.

The composition may further comprise one or more of the following: orotic acid or a salt thereof; carnitine; and coenzyme Q10.

The orotic acid may be present as an orotate salt. The orotate may be selected from, for example, magnesium orotate, zinc orotate, calcium orotate, chromium orotate, potassium orotate, copper orotate, iron orotate, manganese orotate, sodium orotate, choline orotate and lithium orotate. In an exemplary embodiment the orotate is magnesium orotate.

Typically the carnitine is L-carnitine, more typically acetyl-L-carnitine.

The coenzyme Q10 may be in a reduced form, for example as ubiquinol.

The two or more probiotic bacterial strains may comprise at least one Lactobacillus species, at least one Bifidobacterium species and/or at least one Streptococcus species. The Lactobacillus may be L. rhamnosus or L. acidophilus. The L. rhamnosus may be L. rhamnosus GG. The Bifidobacterium may be B. bifidum. The Streptococcus may be S. thermophilus.

The composition may comprise a multi-strain combination of probiotic organisms. In one exemplary embodiment, the combination comprises L. rhamnosus, B. bifidum, and S. thermophilus. In a further exemplary embodiment the combination comprises L. acidophilus, B. bifidum, and S. thermophilus. In accordance with these exemplary embodiments, the combination may further comprise magnesium orotate, coenzyme Q10 (for example as ubiquinol), and optionally L-carnitine (for example as acetyl-L-carnitine).

In one embodiment the composition comprises or consists essentially of L. acidophilus, B. bifidum, S. thermophilus, magnesium orotate and coenzyme Q10.

The composition may further comprise one or more prebiotic components.

In a fifth aspect the present disclosure provides a method for boosting energy levels, the method comprising administering to a subject in need thereof an effective amount of one or more probiotic bacterial strains selected from Lactobacillus rhamnosus, Lactobacillus plantarum, Lactobacillus bulgaricus, Lactobacillus gasseri, Lactobacillus reuteri, Lactobacillus paracasei, Lactobacillus casei, Lactobacillus acidophilus, Lactobacillus fermentum, Lactobacillus salvarius, Lactococcus lactis Streptococcus thermophilus, Bifidobacterium breve, Bifidobacterium bifidum, Bifidobacterium animalis subsp. lactis (B. lactis), Bifidobacterium animalis subsp. animalis (B. animalis), Bifidobacterium infantis, Bifidobacterium longum and Bifidobacterium pseudocatenulatum.

The subject may suffer fatigue and lack of energy resulting from, or associated with, for example, stress, sleep deprivation, diet, age, and/or sore muscles.

The treatment may further comprise administering to the subject an effective amount of one or more of the following: orotic acid or a salt thereof; carnitine; and coenzyme Q10.

The orotic acid may be present as an orotate salt. The orotate may be selected from, for example, magnesium orotate, zinc orotate, calcium orotate, chromium orotate, potassium orotate, copper orotate, iron orotate, manganese orotate, sodium orotate, choline orotate and lithium orotate. In an exemplary embodiment the orotate is magnesium orotate.

Typically the carnitine is L-carnitine, more typically acetyl-L-carnitine.

The coenzyme Q10 may be in a reduced form, for example as ubiquinol.

The one or more probiotic bacterial strains may comprise at least one Lactobacillus species, at least one Bifidobacterium species and/or at least one Streptococcus species. The Lactobacillus may be L. rhamnosus or L. acidophilus. The L. rhamnosus may be L. rhamnosus GG. The Bifidobacterium may be B. bifidum. The Streptococcus may be S. thermophilus.

The method may comprise the administration of a multi-strain combination of probiotic organisms. In one exemplary embodiment, the combination comprises L. rhamnosus, B. bifidum, and S. thermophilus. In a further exemplary embodiment the combination comprises L. acidophilus, B. bifidum, and S. thermophilus. In accordance with these exemplary embodiments, the combination may further comprise magnesium orotate, coenzyme Q10 (for example as ubiquinol), and optionally L-carnitine (for example as acetyl-L-carnitine).

In one embodiment the combination comprises or consists essentially of L. acidophilus, B. bifidum, S. thermophilus, magnesium orotate and coenzyme Q10.

The method may further comprise administration to the subject of one or more prebiotic components. The prebiotic may be administered in the same composition as the probiotic strain(s) or in a different composition.

In a sixth aspect the present disclosure provides a probiotic composition for boosting energy levels, wherein the composition comprises two or more bacterial strains selected from Lactobacillus rhamnosus, Lactobacillus plantarum, Lactobacillus bulgaricus, Lactobacillus gasseri, Lactobacillus reuteri, Lactobacillus paracasei, Lactobacillus casei, Lactobacillus acidophilus, Lactobacillus fermentum, Lactobacillus salvarius, Lactococcus lactis Streptococcus thermophilus, Bifidobacterium breve, Bifidobacterium bifidum, Bifidobacterium animalis subsp. lactis (B. lactis), Bifidobacterium animalis subsp. animalis (B. animalis), Bifidobacterium infantis, Bifidobacterium longum and Bifidobacterium pseudocatenulatum.

The composition may further comprise one or more of the following: orotic acid or a salt thereof; carnitine; and coenzyme Q10.

The orotic acid may be present as an orotate salt. The orotate may be selected from, for example, magnesium orotate, zinc orotate, calcium orotate, chromium orotate, potassium orotate, copper orotate, iron orotate, manganese orotate, sodium orotate, choline orotate and lithium orotate. In an exemplary embodiment the orotate is magnesium orotate.

Typically the carnitine is L-carnitine, more typically acetyl-L-carnitine.

The coenzyme Q10 may be in a reduced form, for example as ubiquinol.

The two or more probiotic bacterial strains may comprise at least one Lactobacillus species, at least one Bifidobacterium species and/or at least one Streptococcus species. The Lactobacillus may be L. rhamnosus or L. acidophilus. The L. rhamnosus may be L. rhamnosus GG. The Bifidobacterium may be B. bifidum. The Streptococcus may be S. thermophilus.

The composition may comprise a multi-strain combination of probiotic organisms. In one exemplary embodiment, the combination comprises L. rhamnosus, B. bifidum, and S. thermophilus. In a further exemplary embodiment the combination comprises L. acidophilus, B. bifidum, and S. thermophilus. In accordance with these exemplary embodiments, the combination may further comprise magnesium orotate, coenzyme Q10 (for example as ubiquinol), and optionally L-carnitine (for example as acetyl-L-carnitine).

In one embodiment the composition comprises or consists essentially of L. acidophilus, B. bifidum, S. thermophilus, magnesium orotate and coenzyme Q10.

The composition may further comprise one or more prebiotic components.

In a seventh aspect the present disclosure provides a method for treating or preventing muscle aches, muscle pain, muscle fatigue and/or muscle degeneration or for promoting muscle repair, the method comprising administering to a subject in need thereof an effective amount of one or more probiotic bacterial strains selected from Lactobacillus rhamnosus, Lactobacillus plantarum, Lactobacillus bulgaricus, Lactobacillus gasseri, Lactobacillus reuteri, Lactobacillus paracasei, Lactobacillus casei, Lactobacillus acidophilus, Lactobacillus fermentum, Lactobacillus salvarius, Lactococcus lactis Streptococcus thermophilus, Bifidobacterium breve, Bifidobacterium bifidum, Bifidobacterium animalis subsp. lactis (B. lactis), Bifidobacterium animalis subsp. animalis (B. animalis), Bifidobacterium infantis, Bifidobacterium longum and Bifidobacterium pseudocatenulatum.

The subject may suffer age-related or injury-related muscle weakness or atrophy. The muscle degeneration may result from, or be associated with, a muscular dystrophy. In exemplary embodiments, the muscle is skeletal muscle.

The treatment may further comprise administering to the subject an effective amount of one or more of the following: orotic acid or a salt thereof; carnitine; and coenzyme Q10.

The orotic acid may be present as an orotate salt. The orotate may be selected from, for example, magnesium orotate, zinc orotate, calcium orotate, chromium orotate, potassium orotate, copper orotate, iron orotate, manganese orotate, sodium orotate, choline orotate and lithium orotate. In an exemplary embodiment the orotate is magnesium orotate.

Typically the carnitine is L-carnitine, more typically acetyl-L-carnitine.

The coenzyme Q10 may be in a reduced form, for example as ubiquinol.

The one or more probiotic bacterial strains may comprise at least one Lactobacillus species, at least one Bifidobacterium species and/or at least one Streptococcus species. The Lactobacillus may be L. rhamnosus or L. acidophilus. The L. rhamnosus may be L. rhamnosus GG. The Bifidobacterium may be B. bifidum. The Streptococcus may be S. thermophilus.

The method may comprise the administration of a multi-strain combination of probiotic organisms. In one exemplary embodiment, the combination comprises L. rhamnosus, B. bifidum, and S. thermophilus. In a further exemplary embodiment the combination comprises L. acidophilus, B. bifidum, and S. thermophilus. In accordance with these exemplary embodiments, the combination may further comprise magnesium orotate, coenzyme Q10 (for example as ubiquinol), and optionally L-carnitine (for example as acetyl-L-carnitine).

In one embodiment the combination comprises or consists essentially of L. acidophilus, B. bifidum, S. thermophilus, magnesium orotate and coenzyme Q10.

The method may further comprise administration to the subject of one or more prebiotic components. The prebiotic may be administered in the same composition as the probiotic strain(s) or in a different composition.

In an eighth aspect the present disclosure provides a probiotic composition for treating or preventing muscle aches, muscle pain, muscle fatigue and/or muscle degeneration or for promoting muscle repair, wherein the composition comprises two or more bacterial strains selected from Lactobacillus rhamnosus, Lactobacillus plantarum, Lactobacillus bulgaricus, Lactobacillus gasseri, Lactobacillus reuteri, Lactobacillus paracasei, Lactobacillus casei, Lactobacillus acidophilus, Lactobacillus fermentum, Lactobacillus salvarius, Lactococcus lactis Streptococcus thermophilus, Bifidobacterium breve, Bifidobacterium bifidum, Bifidobacterium animalis subsp. lactis (B. lactis), Bifidobacterium animalis subsp. animalis (B. animalis), Bifidobacterium infantis, Bifidobacterium longum and Bifidobacterium pseudocatenulatum.

The composition may further comprise one or more of the following: orotic acid or a salt thereof; carnitine; and coenzyme Q10.

The orotic acid may be present as an orotate salt. The orotate may be selected from, for example, magnesium orotate, zinc orotate, calcium orotate, chromium orotate, potassium orotate, copper orotate, iron orotate, manganese orotate, sodium orotate, choline orotate and lithium orotate. In an exemplary embodiment the orotate is magnesium orotate.

Typically the carnitine is L-carnitine, more typically acetyl-L-carnitine.

The coenzyme Q10 may be in a reduced form, for example as ubiquinol.

The two or more probiotic bacterial strains may comprise at least one Lactobacillus species, at least one Bifidobacterium species and/or at least one Streptococcus species. The Lactobacillus may be L. rhamnosus or L. acidophilus. The L. rhamnosus may be L. rhamnosus GG. The Bifidobacterium may be B. bifidum. The Streptococcus may be S. thermophilus.

The composition may comprise a multi-strain combination of probiotic organisms. In one exemplary embodiment, the combination comprises L. rhamnosus, B. bifidum, and S. thermophilus. In a further exemplary embodiment the combination comprises L. acidophilus, B. bifidum, and S. thermophilus. In accordance with these exemplary embodiments, the combination may further comprise magnesium orotate, coenzyme Q10 (for example as ubiquinol), and optionally L-carnitine (for example as acetyl-L-carnitine).

In one embodiment the composition comprises or consists essentially of L. acidophilus, B. bifidum, S. thermophilus, magnesium orotate and coenzyme Q10.

The composition may further comprise one or more prebiotic components.

Compositions according to the above aspects are typically in a form suitable for oral administration. The composition may be a solid or liquid composition. The composition may be in unit dosage form. In one particular embodiment, the unit dosage form is a capsule. The composition may be in the form of a beverage or food supplement.

In accordance with methods of the present disclosure probiotic compositions may be administered to subjects in need thereof as food or drink supplements. The compositions may be also be administered as an adjunct to one or more other treatments or therapies for treating or preventing fatigue, promoting synthesis of adenosine triphosphate (ATP) and/or boosting energy levels, for treating or preventing muscle aches, muscle pain, muscle fatigue and/or muscle degeneration or for promoting muscle repair.

Also provided herein is the use of one or more probiotic bacterial strains selected from Lactobacillus rhamnosus, Lactobacillus plantarum, Lactobacillus bulgaricus, Lactobacillus gasseri, Lactobacillus reuteri, Lactobacillus paracasei, Lactobacillus casei, Lactobacillus acidophilus, Lactobacillus fermentum, Lactobacillus salvarius, Lactococcus lactis Streptococcus thermophilus, Bifidobacterium breve, Bifidobacterium bifidum, Bifidobacterium animalis subsp. lactis (B. lactis), Bifidobacterium animalis subsp. animalis (B. animalis), Bifidobacterium infantis, Bifidobacterium longum and Bifidobacterium pseudocatenulatum, for the manufacture of a probiotic composition for treating or preventing fatigue, for promoting synthesis of adenosine triphosphate (ATP) and/or boosting energy levels, for treating or preventing muscle aches, muscle pain, muscle fatigue and/or muscle degeneration or for promoting muscle repair.

### BRIEF DESCRIPTION OF THE FIGURES

FIG. 1 shows an HPLC chromatogram of red blood cell extract with and without alkaline phosphatase, prepared and analysed according to Example 4 (injected sample volume (5 μl) is equivalent to approx. 1.2 μl of packaged red blood cells).

FIG. 2 shows an HPLC chromatogram of red blood cell extract with and without alkaline phosphatase, prepared and analysed according to Example 4 (injected sample volume (10 μl) is equivalent to approx. 2.4 μl of packaged red blood cells).

### DETAILED DESCRIPTION

Unless defined otherwise, all technical and scientific terms used herein have the same meaning as commonly understood by those of ordinary skill in the art to which the disclosure belongs. Although any methods and materials similar or equivalent to those described herein can be used in the practice or testing of the present disclosure, typical methods and materials are described.

The articles “a” and “an” are used herein to refer to one or to more than one (i.e., to at least one) of the grammatical object of the article. By way of example, “an element” means one element or more than one element.

In the context of this specification, the term “about,” is understood to refer to a range of numbers that a person of skill in the art would consider equivalent to the recited value in the context of achieving the same function or result.

Throughout this specification and the claims which follow, unless the context requires otherwise, the word “comprise”, and variations such as “comprises” or “comprising”, will be understood to imply the inclusion of a stated integer or step or group of integers or steps but not the exclusion of any other integer or step or group of integers or steps.

In the context of this specification, the term “probiotic” is to be given its broadest construction and is understood to refer to a microbial cell population or preparation, or component of a microbial cell population or preparation, which when administered to a subject in an effective amount promotes a health benefit in the subject.

In the context of this specification, the term “prebiotic” is to be given its broadest construction and is understood to refer to any non-digestible substance that stimulates the growth and/or activity of bacteria in the digestive system.

In the context of this specification, the terms “food”, “foods”, “beverage” or “beverages” include but are not limited to health foods and beverages, functional foods and beverages, and foods and beverages for specified health use. When such foods or beverages of the present invention are used for subjects other than humans, the terms can be used to include a feedstuff.

The term “subject” as used herein refers to any mammal, including, but not limited to, livestock and other farm animals (such as cattle, goats, sheep, horses, pigs and chickens), performance animals (such as racehorses), companion animals (such as cats and dogs), laboratory test animals and humans. Typically the subject is a human.

As used herein, the term “effective amount” refers to an amount of a composition that is sufficient to effect one or more beneficial or desired outcomes. An “effective amount” can be provided in one or more administrations. The exact amount required will vary depending on factors such as the identity and number of individual probiotic strains employed in the composition, the subject being treated, the nature and degree of any disease or condition suffered by the subject and the age and general health of the subject, and the form in which the composition is administered. Thus, it is not possible to specify an exact “effective amount”. However, for any given case, an appropriate “effective amount” may be determined by one of ordinary skill in the art using only routine experimentation.

As used herein the terms “treating”, “treatment” and the like refer to any and all applications which remedy, or otherwise hinder, retard, or reverse the progression of, an infection or disease or at least one symptom of an infection or disease, including reducing the severity of an infection or disease. Thus, treatment does not necessarily imply that a subject is treated until complete elimination of the infection or recovery from a disease. Similarly, the terms “preventing”, “prevention” and the like refer to any and all applications that prevent the establishment of an infection or disease or otherwise delay the onset of an infection or disease.

As used herein, the terms “promote”, “promoting” and the like in the context of promoting synthesis of adenosine triphosphate (ATP), or promoting muscle repair means that a composition is administered to, or a method is used for, a subject for a period effective to stimulate, promote or improve the synthesis of adenosine triphosphate (ATP), or promote or improve repair of muscles, directly or indirectly, as determined by comparison with the extent, amount or degree of synthesis of adenosine triphosphate (ATP), or muscle repair in subjects not being administered the composition or using the method. Any suitable method(s) of assessing synthesis of adenosine triphosphate (ATP) or muscle repair can be used to determine whether a promotion, stimulation or improvement occurs, as will be readily appreciated by those skilled in the art.

As used herein, the terms “boost”, “boosting” and the like in the context of boosting energy levels refer to the ability to achieve a measurable increase or improvement in the energy levels in a subject, or otherwise prevent, hinder, retard, or reverse the deterioration of energy levels in any way whatsoever. Thus the terms “boost”, “boosting” and the like are to be considered in their broadest context.

The term “optionally” is used herein to mean that the subsequently described feature may or may not be present or that the subsequently described event or circumstance may or may not occur. Hence the specification will be understood to include and encompass embodiments in which the feature is present and embodiments in which the feature is not present, and embodiments in which the event or circumstance occurs as well as embodiments in which it does not.

Provided herein are methods for treating or preventing fatigue, comprising administering to a subject in need thereof an effective amount of one or more probiotic bacterial strains selected from Lactobacillus rhamnosus, Lactobacillus plantarum, Lactobacillus bulgaricus, Lactobacillus gasseri, Lactobacillus reuteri, Lactobacillus paracasei, Lactobacillus casei, Lactobacillus acidophilus, Lactobacillus fermentum, Lactobacillus salvarius, Lactococcus lactis Streptococcus thermophilus, Bifidobacterium breve, Bifidobacterium bifidum, Bifidobacterium animalis subsp. lactis (B. lactis), Bifidobacterium animalis subsp. animalis (B. animalis), Bifidobacterium infantis, Bifidobacterium longum and Bifidobacterium pseudocatenulatum.

Also provided are methods for promoting synthesis of adenosine triphosphate (ATP), comprising administering to a subject in need thereof an effective amount of one or more probiotic bacterial strains selected from Lactobacillus rhamnosus, Lactobacillus plantarum, Lactobacillus bulgaricus, Lactobacillus gasseri, Lactobacillus reuteri, Lactobacillus paracasei, Lactobacillus casei, Lactobacillus acidophilus, Lactobacillus fermentum, Lactobacillus salvarius, Lactococcus lactis Streptococcus thermophilus, Bifidobacterium breve, Bifidobacterium bifidum, Bifidobacterium animalis subsp. lactis (B. lactis), Bifidobacterium animalis subsp. animalis (B. animalis), Bifidobacterium infantis, Bifidobacterium longum and Bifidobacterium pseudocatenulatum.

Also provided are methods for boosting energy levels, comprising administering to a subject in need thereof an effective amount of one or more probiotic bacterial strains selected from Lactobacillus rhamnosus, Lactobacillus plantarum, Lactobacillus bulgaricus, Lactobacillus gasseri, Lactobacillus reuteri, Lactobacillus paracasei, Lactobacillus casei, Lactobacillus acidophilus, Lactobacillus fermentum, Lactobacillus salvarius, Lactococcus lactis Streptococcus thermophilus, Bifidobacterium breve, Bifidobacterium bifidum, Bifidobacterium animalis subsp. lactis (B. lactis), Bifidobacterium animalis subsp. animalis (B. animalis), Bifidobacterium infantis, Bifidobacterium longum and Bifidobacterium pseudocatenulatum.

Also provided are methods for treating or preventing muscle aches, muscle pain, muscle fatigue and/or muscle degeneration or for promoting muscle repair, the method comprising administering to a subject in need thereof an effective amount of one or more probiotic bacterial strains selected from Lactobacillus rhamnosus, Lactobacillus plantarum, Lactobacillus bulgaricus, Lactobacillus gasseri, Lactobacillus reuteri, Lactobacillus paracasei, Lactobacillus casei, Lactobacillus acidophilus, Lactobacillus fermentum, Lactobacillus salvarius, Lactococcus lactis Streptococcus thermophilus, Bifidobacterium breve, Bifidobacterium bifidum, Bifidobacterium animalis subsp. lactis (B. lactis), Bifidobacterium animalis subsp. animalis (B. animalis), Bifidobacterium infantis, Bifidobacterium longum and Bifidobacterium pseudocatenulatum. The subject may suffer age-related or injury-related muscle weakness or atrophy. The muscle degeneration may result from, or be associated with, a muscular dystrophy. In exemplary embodiments, the muscle is skeletal muscle.

In particular embodiments of the present disclosure, the methods also comprise the administration of orotic acid or a salt thereof. Suitable salts of orotic acid for use in accordance with embodiments of the present disclosure include, but are not limited to, magnesium orotate, zinc orotate, calcium orotate, chromium orotate, potassium orotate, copper orotate, iron orotate, manganese orotate, sodium orotate, choline orotate and lithium orotate. Those skilled in the art will appreciate that other salts may also be employed and the scope of the present disclosure is not limited by reference to any particular salt.

In particular embodiments of the present disclosure, the methods also comprise the administration of carnitine. In particular embodiments the carnitine is L-carnitine. In particular embodiments the L-carnitine is acetyl-L-carnitine. In particular embodiments of the present disclosure, the methods also comprise the administration of coenzyme Q10. The coenzyme Q10 may, for example be provided in a reduced form, such as ubiquinol.

In accordance with methods of the present disclosure, the one or more probiotic strains may be present in a composition as specially selected strains as a culture concentrate or as part of a multi-strain blend, optionally with a variety of excipients, and optionally further comprising orotic acid or a salt thereof, carnitine and/or coenzyme Q10. Accordingly, novel probiotic compositions for treating or preventing fatigue, for promoting synthesis of adenosine triphosphate (ATP), for muscle repair and for treating muscle aches, muscle pain or muscle fatigue, and/or for boosting energy levels are provided herein. The probiotic compositions may also improve the health of a subject by supporting healthy energy production, supporting cardiovascular and heart function, and/or supporting healthy mitochondrial function. Probiotic compositions of the present disclosure typically comprise strains of two or more bacterial species selected from Lactobacillus rhamnosus, Lactobacillus plantarum, Lactobacillus bulgaricus, Lactobacillus gasseri, Lactobacillus reuteri, Lactobacillus paracasei, Lactobacillus casei, Lactobacillus acidophilus, Lactobacillus fermentum, Lactobacillus salvarius, Lactococcus lactis Streptococcus thermophilus, Bifidobacterium breve, Bifidobacterium bifidum, Bifidobacterium animalis subsp. lactis (B. lactis), Bifidobacterium animalis subsp. animalis (B. animalis), Bifidobacterium infantis, Bifidobacterium longum and Bifidobacterium pseudocatenulatum.

The methods may comprise the administration of a multi-strain combination of probiotic organisms. In one exemplary embodiment, the combination comprises L. rhamnosus, B. bifidum, and S. thermophilus. In a further exemplary embodiment the combination comprises L. acidophilus, B. bifidum, and S. thermophilus. In accordance with these exemplary embodiments, the combination may further comprise magnesium orotate, coenzyme Q10 (for example as ubiquinol), and optionally L-carnitine (for example as acetyl-L-carnitine).

In a particular embodiment the multi-strain combination comprises L. acidophilus, B. bifidum, S. thermophilus and the combination further comprises magnesium orotate and coenzyme Q10.

Probiotic compositions of the present disclosure may further comprise, and methods disclosed herein may further comprise the administration of, a yeast strain such as Saccharomyces cerevisiae or Saccharomyces boulardii.

In accordance with the present disclosure subjects to be treated may suffer fatigue resulting from a variety of causes, or associated with a variety of conditions or other symptoms. By way of example, the fatigue may result from, or be associated with stress, sleep deprivation, diet, age, and/or sore muscles.

Whilst not wishing to be bound by theory the probiotic compositions may treat or prevent fatigue and boost energy levels by supporting the vital function of mitochondria in cells by promoting synthesis of adenosine triphosphate (ATP) and supporting healthy cardiovascular function. Largely found in the heart, muscles and the brain, mitochondria are essential organelles for the metabolism, processing oxygen and substances from foods into energy. Poorly functioning mitochondria can result in fatigue, lack of concentration and sore muscles.

In accordance with the present disclosure subjects to be treated may suffer muscle degeneration resulting from a variety of causes, or associated with a variety of conditions or other symptoms. By way of example, the muscle degeneration may result from, or be associated with age-related or injury-related muscle weakness or atrophy, or a muscular dystrophy. Exemplary muscular dystrophies include Duchenne muscular dystrophy, distal muscular dystrophy, Becker's muscular dystrophy, congenital muscular dystrophy, myotonic muscular dystrophy, limb-girdle muscular dystrophy and Emery-Dreifuss muscular dystrophy.

The amounts of individual microbial strains to be administered to subjects or to be included in compositions disclosed herein will depend on a variety of factors including the identity and number of individual strains employed, the condition or disease to be treated or against which the composition is designed to be used, and the form in which a composition is administered. For any given case, appropriate amounts may be determined by one of ordinary skill in the art using only routine experimentation. By way of example only, the amount of each microbial strain present in a single dose of a composition disclosed herein may be from about 1×10² cfu to about 1×10¹¹ cfu, and may be about 1×10³ cfu, about 2.5×10³ cfu, about 5×10³ cfu, about 7.5×10³ cfu, 1×10⁴ cfu, about 2.5×10⁴ cfu, about 5×10⁴ cfu, about 7.5×10⁴ cfu, about 1×10⁵ cfu, about 2.5×10⁵ cfu, about 5×10⁵ cfu, about 7.5×10⁵ cfu, about 1×10⁶ cfu, about 2.5×10⁶ cfu, about 5×10⁶ cfu, about 7.5×10⁶ cfu, about 1×10⁷ cfu, about 2.5×10⁷ cfu, about 5×10⁷ cfu, about 7.5×10⁷ cfu, about 1×10⁸ cfu, about 2.5×10⁸ cfu, about 5×10⁸ cfu, about 7.5×10⁸ cfu, about 1×10⁹ cfu, about 2.5×10⁹ cfu, about 5×10⁹ cfu, about 7.5×10⁹ cfu, about 1×10¹⁰ cfu, about 2.5×10¹⁰ cfu, about 5×10¹⁰ cfu, about 7.5×10¹⁰ cfu, and about 1×10¹¹ cfu.

In exemplary embodiments, each strain is present in an amount of between about 1×10⁹ and about 10×10⁹ cfu per dose of the composition. Thus, in one embodiment, the composition comprises about 10 billion cfu of L. acidophilus, B. bifidum and S. thermophilus per dose. Typically, the composition comprises about 5 billion cfu L. acidophilus, about 2 billion cfu B. bifidum, and about 3 billion cfu S. thermophilus.

Also contemplated by the present disclosure are variants of the microbial strains described herein. As used herein, the term “variant” refers to both naturally occurring and specifically developed variants or mutants of the microbial strains disclosed and exemplified herein. Variants may or may not have the same identifying biological characteristics of the specific strains exemplified herein, provided they share similar advantageous properties in terms of their ability to be used as probiotic strains for the treatment or prevention of kidney disease, dysbiotic gut or compositions associated with dysbiotic gut. Illustrative examples of suitable methods for preparing variants of the microbial strains exemplified herein include, but are not limited to, culturing under selective growth conditions, gene integration techniques such as those mediated by insertional elements or transposons or by homologous recombination, other recombinant DNA techniques for modifying, inserting, deleting, activating or silencing genes, intraspecific protoplast fusion, mutagenesis by irradiation with ultraviolet light or X-rays, or by treatment with a chemical mutagen such as nitrosoguanidine, methylmethane sulfonate, nitrogen mustard and the like, and bacteriophage-mediated transduction. Suitable and applicable methods are well known in the art and are described, for example, in J. H. Miller, Experiments in Molecular Genetics, Cold Spring Harbor Laboratory Press, Cold Spring Harbor, N.Y. (1972); J. H. Miller, A Short Course in Bacterial Genetics, Cold Spring Harbor Laboratory Press, Cold Spring Harbor, N.Y. (1992); and J. Sambrook, D. Russell, Molecular Cloning: A Laboratory Manual, 3rd ed., Cold Spring Harbor Laboratory Press, Cold Spring Harbor, N.Y. (2001), inter alia.

Also encompassed by the term “variant” as used herein are microbial strains phylogenetically closely related to strains disclosed herein and strains possessing substantial sequence identity with the strains disclosed herein at one or more phylogenetically informative markers such as rRNA genes, elongation and initiation factor genes, RNA polymerase subunit genes, DNA gyrase genes, heat shock protein genes and recA genes. For example, the 16S rRNA genes of a “variant” strain as contemplated herein may share about 85%, 86%_{,} 87%_{,} 88%_{,} 89%_{,} 90%_{,} 91%, 92%, 93%, 94%, 95%, 96%, 97%, 98% or 99% sequence identity with a strain disclosed herein.

The bacterial strains to be employed in accordance with the present disclosure may be cultured according to any suitable method known to the skilled addressee and may be prepared for addition to a composition by, for example, freeze-drying, spray-drying or lyophilisation. Thus, in embodiments of the present disclosure the bacterial strains may be in a dried form (such as lyophilized or sporulated form) in a suitable carrier medium, for example a FOS medium or other soluble fiber, sugar, nutrient or base material for the composition, with which the bacterial strains can be presented in an orally administrable form. One or more of the strains may be encapsulated in, for example, a suitable polymeric matrix to improve long term stability and storage of the compositions. In one example, encapsulation may comprise alginate beads, although those skilled in the art will appreciate that any suitable encapsulation material or matrix may be used. Encapsulation may be achieved using methods and techniques known to those skilled in the art.

Effective amounts orotic acid or salt thereof, carnitine, and coenzyme Q10 may each range from about 50 mg to about 4000 mg per day for a human subject. In certain embodiments, about 200 mg to about 4000 mg of orotic acid or salt thereof, or carnitine, or about 50 mg to 200 mg of coenzyme Q10 per day is useful, e.g., about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, about 2000 mg, about 3000 mg or about 4000 mg, of orotic acid or salt thereof, or carnitine per day; and about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg or about 200 mg of coenzyme Q10 per day. In certain embodiments, orotic acid or salt thereof, carnitine, and/or coenzyme Q10 are provided in a range of between about 200 mg to about 4000 mg, between about 200 mg to about 2000 mg, between about 400 mg to about 2000 mg, between 400 mg to about 1000 mg, between about 500 mg to about 2000 mg, between about 600 mg to about 2000 mg, between about 700 mg to about 2000 mg, between about 800 mg to about 2000 mg, between about 800 mg to about 1600 mg, between about 800 to about 1500 mg, between about 800 mg to about 1400 mg, between about 800 mg to about 1300 mg, between about 800 mg to about 1200 mg, between about 800 mg to about 1100 mg, between about 800 mg to about 1000 mg, or between about 800 mg to about 900 mg, inclusive. In certain embodiments, the orotic acid or salt thereof, carnitine, and/or coenzyme Q10 are provided in a range of between about 800 mg to about 1600 mg. In certain embodiments, the amount orotic acid or salt thereof, carnitine, and/or coenzyme Q10 administered per day is about 800 mg, about 1200 mg or about 1600 mg. In certain embodiments, coenzyme Q10 is provided in a range of between about 50 mg to about 200 mg, or between about 50 mg to about 100 mg. In certain embodiments, the amount coenzyme Q10 administered per day is about 50 mg, about 55 mg, about 60 mg, about 65 mg, about 70 mg, about 75 mg, about 80 mg, about 85 mg, about 90 mg or about 100 mg. The amounts to be administered to subjects or to be included in compositions disclosed herein will depend on a variety of factors including the identity of the compound or agent employed, the nature and extent of any condition suffered by the subject, and the form in which a composition is administered. For any given case, appropriate amounts may be determined by one of ordinary skill in the art using only routine experimentation.

In accordance with methods disclosed herein the orotic acid or salt thereof, carnitine, coenzyme Q10 and the one or more probiotic strains may be coadministered sequentially or simultaneously, and as part of the same or different compositions. Moreover the orotic acid or salt thereof, carnitine, coenzyme Q10 and probiotic strain(s) may be coadministered with one or more other treatments or therapies for the treatment or prevention of fatigue, for promoting synthesis of adenosine triphosphate (ATP), for the treatment or prevention of muscle degeneration, muscle aches, muscle pain, muscle fatigue or for promoting muscle repair and/or for boosting energy levels. By “coadministered” is meant simultaneous administration in the same formulation or in two different formulations via the same or different routes or sequential administration by the same or different routes. By “sequential” administration is meant a time difference of from seconds, minutes, hours or days between the administration of the agents, compositions or treatments. Sequential administration may be in any order. Thus, in accordance with embodiments of the present disclosure, the orotic acid or salt thereof, carnitine, coenzyme Q10 and the probiotic strain(s) may be formulated in separate unit dosage forms intended to be coadministered. The unit dosage forms may be, for example, capsules. In an exemplary embodiment, the probiotic composition may be administered in a daily dose of 100 mg of a multi-strain combination (10 billion CFU bacteria) and an effective amount of orotic acid or salt thereof, carnitine, coenzyme Q10, to be taken, for example, as 2×50 mg probiotic capsules and 2× capsules comprising one or more of orotic acid or salt thereof, carnitine and coenzyme Q10 (‘compound’ capsules) in the morning, followed by 2×500 mg probiotic capsules and 2× compound capsules at night (8 capsules per day). A package may contain blister packs of probiotic and compound capsules, each blister pack providing one, two or more days supply. Those skilled in the art will also appreciate that unit dosage forms comprising the orotic acid or salt thereof, carnitine, coenzyme Q10 and the probiotic strain(s) need not be of the same type.

In some embodiments, methods of the present disclosure may comprise administration to the subject of one or more prebiotic components. Similarly, in some embodiments, compositions comprising one or more probiotic strains may further comprise at least one prebiotic component. Suitable prebiotics include polydextrose, inulin, fructooligosaccharides (FOS), xylooligosaccharides (XOS), galactooligosaccharides (GOS), mannan oligosaccharides, protein-based green lipped mussel extract, and various prebiotic-containing foods such as raw onion, raw leek, raw chickory root and raw artichoke. In certain embodiments the prebiotic component is a fructooligosaccharide. Those skilled in the art will appreciate that other sources of fiber and/or prebiotics may be added to the compositions.

In accordance with particular embodiments of the invention the at least one prebiotic component may be administered or be present in a composition in an amount of from about 1 mg to about 100 g, or more typically between about 5 mg to about 50 g. Alternatively, the composition may comprise about 10 mg, 100 mg, 1 g, 5 g, 10 g, 15 g, 20 g, 25 g, 30 g, 35 g, 40 g or 45 g of prebiotic.

Molecules such as alpha-lactalbumin, beta-lactoglobulin, glycomacropeptides, immunoglobulin G, and bovine serum albumin may augment the health of the gut individually and/or in combination with each other as well as with the administration of probiotic bacteria, and optionally a prebiotic, as disclosed herein. Accordingly, embodiments of the present invention contemplate the addition of a protein-containing component to compositions disclosed herein, and the administration thereof to subjects in accordance with methods of the invention. The protein-containing component may comprise a protein powder, such as a milk powder. The milk powder may be skim milk powder. The protein-containing component may comprise one or more of alpha-lactalbumin, beta-lactoglobulin, glycomacropeptides, immunoglobulin G and/or bovine serum albumin.

Also contemplated herein are packages comprising a probiotic composition in one unit dosage form and a prebiotic in a separate unit dosage form, wherein the separate unit dosage forms are intended to be coadministered. The unit dosage forms may be, for example, capsules. In an exemplary embodiment, the probiotic composition may be administered in a daily dose of 2000 mg of a multi-strain combination (200 billion CFU bacteria) and 3000 mg prebiotic (such as FOS), to be taken, for example, as 2×50 mg probiotic capsules and 2×750 mg prebiotic capsules in the morning, followed by 2×500 mg probiotic capsules and 2×750 mg prebiotic capsules at night (8 capsules per day). A package may contain blister packs of probiotic and prebiotic capsules, each blister pack providing one, two or more days supply.

The compositions comprising one or more probiotic strains may further comprise vitamins and/or minerals and/or amino acids. The vitamins and minerals may be selected from, but not limited to: vitamins A, B₁, B₂, B₃, B₅, B₆, B₉, B₁₂, C, D, E and calcium, chromium, copper, fluorine, iodine, iron, magnesium, manganese, molybdenum, phosphorus, potassium, selenium, sodium and zinc. The amino acids may be selected from, but are not limited to: alanine, arginine, aspartic acid, cystine, glycine, histidine, lysine, methionine, phenylalanine, proline, serine, threonine, tryptophan and tyrosine.

Compositions of the invention may further comprise one or more antioxidants. The antioxidants may be water soluble or lipid soluble antioxidants. Exemplary water soluble antioxidants include sodium ascorbate, calcium ascorbate, potassium ascorbate, ascorbic acid, glutathione, lipoic acid and uric acid. Exemplary lipid soluble antioxidants include tocopherols, tocotrienols, phenols, polyphenols and the like.

The compositions may additionally include any suitable additives, carriers, additional therapeutic agents, bioavailability enhancers, side-effect suppressing components, diluents, buffers, flavouring agents, binders, preservatives or other ingredients that are not detrimental to the efficacy of the composition. In some embodiments, the probiotic strains may comprise from about 50% to about 90% by weight of the composition, based on the total weight of the composition including a carrier medium, or from about 60% to about 80% by weight of the composition.

Compositions of the invention can be readily manufactured by those skilled in the art using known techniques and processes. For example, the bacterial strains can be seeded from standard stock into a reactor and grown in standardized media until a predetermined CFU/g concentration is reached. The bulk material can then be drained from the reactor and dried by spray drying, lyophilization, or flatbed oven drying. The dried bacterial material can then be blended with the carrier medium and the resulting mixture can be pressed into tablets, filled into foil pouches as a granular solid, or introduced into gelatin capsules as a particulate material.

Compositions of the present disclosure may be suitably formulated for oral administration, and may be prepared according to conventional methods well known in the pharmaceutical and nutraceutical industries, such as those described in Remington's Pharmaceutical Handbook (Mack Publishing Co., NY, USA) using suitable excipients, diluents and fillers. Exemplary additional ingredients include citric acid, magnesium oxide, silicon dioxide, etc. In general, oral compositions are prepared by uniformly and intimately bringing into association the components of the composition with a liquid carrier or finely divided solid carrier, or both and then, if necessary, shaping the product into the desired composition. Oral dosage forms may include soluble sachets, orally soluble forms, capsules, tablets, chewable tablets, multi-layer tablets with, for example, time- and/or pH-dependent release, and granulates.

Compositions suitable for oral administration may be presented as discrete units (i.e. dosage forms) such as gelatine or HPMC capsules, cachets or tablets, each containing a predetermined amount of each component of the composition as a powder, granules, as a solution or a suspension in an aqueous liquid or a non-aqueous liquid, or as an oil-in-water liquid emulsion or a water-in-oil liquid emulsion.

When the composition is formulated as capsules, the components of the composition may be formulated with one or more pharmaceutically acceptable carriers such as starch, lactose, microcrystalline cellulose and/or silicon dioxide. Additional ingredients may include lubricants such as magnesium stearate and/or calcium stearate. The capsules may optionally be coated, for example, with a film coating or an enteric coating and/or may be formulated so as to provide slow or controlled release of the composition therein.

Tablets may be prepared by compression or moulding, optionally with one or more accessory ingredients. Compressed tablets may be prepared by compressing in a suitable machine the components of the composition in a free-flowing form such as a powder or granules, optionally mixed with a binder, lubricant (for example magnesium stearate or calcium stearate), inert diluent or a surface active/dispersing agent. Moulded tablets may be made by moulding a mixture of the powdered composition moistened with an inert liquid diluent, in a suitable machine. The tablets may optionally be coated, for example, with a film coating or an enteric coating and/or may be formulated so as to provide slow or controlled release of the composition therein.

The compositions may be provided to the user in a powder form. The compositions may be added in powder form by the user to any type of drink or food product (for example water, fruit juice or yoghurt) and consumed there after. In another embodiment, the compositions may simply be consumed as a powder in the absence of a drink or additional food product.

The one or more probiotic strains may be conveniently incorporated in a variety of food and/or beverage products, nutraceutical products, probiotic supplements, food additives, pharmaceuticals and over-the-counter formulations. The food or food additive may be a solid form such as a powder, or a liquid form. Specific examples of the types of beverages or foods include, but are not limited to water-based, milk-based, yoghurt-based, other dairy-based, milk-substitute based such as soy milk or oat milk, or juice-based beverages, water, soft drinks, carbonated drinks, and nutritional beverages, (including a concentrated stock solution of a beverage and a dry powder for preparation of such a beverage); baked products such as crackers, breads, muffins, rolls, bagels, biscuits, cereals, bars such as muesli bars, health food bars and the like, dressings, sauces, custards, yoghurts, puddings, pre-packaged frozen meals, soups and confectioneries.

Those skilled in the art will appreciate that single or multiple administrations of compositions disclosed herein can be carried out with dose levels and dosing regimes being determined as required depending on the circumstances and on the condition of the subject to be treated. Suitable dosage regimes can readily be determined by the skilled addressee. A broad range of doses may be applicable. Dosage regimens may be adjusted to provide the optimum therapeutic response. Those skilled in the art will appreciate that the exact amounts and rates of administration of the probiotic microorganisms will depend on a number of factors such as the particular composition being administered, the age, body weight, general health, sex and dietary requirements of the subject, as well as any drugs or agents used in combination or coincidental with the compositions. For example, several divided doses may be administered hourly, daily, weekly, monthly or at other suitable time intervals or the dose may be proportionally reduced as indicated by the exigencies of the situation. Based on the teaching herein those skilled in the art will, by routine trial and experimentation, be capable of determining suitable dosage regimes on a case-by-case basis.

In general, compositions of the present disclosure may be administered in any suitable dose amount that is effective as a health supplement, food supplement, food additive, and/or therapeutic agent to achieve the desired health outcome. In some embodiments, an effective dose of a composition may be in a range of from 1 g to 15 g for an adult subject, or between about 2 g and about 10 g. Pediatric dosages may be in the range of 15% to 90% of adult dosages. In therapeutic applications a constant dosage of the composition can be administered over time, for example, about 2 g to about 4 g per day, up to about 6 g to about 10 g per day, depending on the severity of the disease or condition to be treated. Once the disease or condition has been effectively ameliorated, the subject can in many instances decrease the dosage to about 2 g to about 4 g per day for maintenance purposes.

By way of example only, a suitable dose may be 2 capsules daily (as required) or twice daily (as required). Those skilled in the art will appreciate that the number of capsules and the frequency of consumption may be varied according to requirements of the individual subject. Suitable dosages, frequency of administration of doses and length of time that administration is continued may be determined in consultation with a healthcare practitioner.

Compositions and methods of the present disclosure may be employed as an adjunct to other therapies or treatments for fatigue, muscle damage, muscle degeneration, or muscle ache, pain or fatigue, for the synthesis of adenosine triphosphate (ATP) and/or for boosting energy levels. Accordingly compositions and methods disclosed herein may be coadministered with other agents that may facilitate a desired therapeutic outcome. By “coadministered” is meant simultaneous administration in the same formulation or in two different formulations via the same or different routes or sequential administration by the same or different routes. By “sequential” administration is meant a time difference of from seconds, minutes, hours or days between the administration of the agents, compositions or treatments. Sequential administration may be in any order.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

The present disclosure will now be described with reference to the following specific examples, which should not be construed as in any way limiting the scope of the invention.

### EXAMPLES

The following examples are illustrative of the invention and should not be construed as limiting in any way the general nature of the disclosure of the description throughout this specification.

### Example 1

### Oral Composition

An exemplary probiotic composition according to the present disclosure has the following ingredients:

### Probiotic Components:

| | |
|---|---|
| Lactobacillus acidophilus | 5 billion CFU/capsule |
| Bifidobacterium bifidum | 2 billion CFU/capsule |
| Streptococcus thermophilus | 3 billion CFU/capsule |

### Other Active Components:

| | | |
|---|---|---|
| Acetyl-L-carnitine | 250 | mg |
| Orotate [as magnesium orotate salt] | 800 | mg |
| coenzyme Q10 [as ubiquinol] | 75 | mg |

### Carrier Components:

magnesium oxide
magnesium gluconate
glutathione

### FOS

### Fructose

### Additional Excipients:

anhydrous citric acid
flavouring
colouring
Product may be formulated as capsule, sachet or other delivery means.

### Example 2

### Oral Capsule Composition

An exemplary probiotic composition according to the present disclosure has the following ingredients, per 2 capsule dose:

### Probiotic Components:

| | |
|---|---|
| Lactobacillus acidophilus | |
| Bifidobacterium bifidum | |
| Streptococcus thermophilus | Total: 10 billion CFU |

### Other Active Components:

| | |
|---|---|
| Magnesium (as magnesium orotate 800 mg) | 50 mg |
| Coenzyme Q10 | 75 mg |

Product is formulated as a capsule. Typically two capsules are taken once daily.

### Example 3

### Oral Capsule Composition

An exemplary probiotic composition according to the present disclosure has the following ingredients, per 2 capsule dose:

### Probiotic Components:

| | | |
|---|---|---|
| Lactobacillus acidophilus | 5 | billion CFU |
| Bifidobacterium bifidum | 2 | billion CFU |
| Streptococcus thermophilus | 3 | billion CFU |
| Total | 10 | billion CFU |

### Other Active Components:

| | | |
|---|---|---|
| Magnesium orotate | 880 | mg |
| Coenzyme Q10 | 85.1 | mg |

Product is formulated as a capsule. Typically two capsules are taken once daily.

### Example 4

### Measurement of Uridine in Red Blood Cells

A method was developed to analyse free (without alkaline phosphatase digestion) and total (with alkaline phosphatase digestion) uridine in red blood cells (RBC) isolated from subjects.

### Collection of Blood Cells

Peripheral (venous) blood (approximately 5 mL) was collected into heparinized tubes and immediately placed on ice. Two aliquots (2 mL each) were transferred into new cooled tubes. Red blood cells were collected by centrifugation (20 min, 4000 rpm, 4° C.), and the cell sediments (1 mL) stored at −80° C.

### Preparation of Red Blood Cell Extractions

Ice-cold MilliQ water (1.25 mL) was added into thawed red blood cell sediment (1 mL). After homogenizing the cell suspension by vortexing, 2 M ice-cold perchloric acid (750 μL, Sigma 311421) was added. The mixture was then centrifuged (20 min, 4000 rpm, 4° C.) and the supernatant (1.35 mL) collected into a cooled 2 mL tube. Ice-cold 2.5 M potassium carbonate (150 μL, Sigma P5833) was added to the supernatant, and the mixture kept on ice for 20 to 30 min. The supernatant was centrifuged (1 min, 14000 g, 4° C.) and aliquoted (200 μL) into four separate tubes and supplemented as follows:
1) 200 μL red blood cell extract+50 μL alkaline phosphatase buffer (100 mM glycine, Sigma G7162; 1 mM magnesium chloride, Sigma M8266; pH adjusted to 9.0)
2) 200 μL red blood cell extract+50 μL alkaline phosphatase buffer containing 200 units of alkaline phosphatase (Sigma P0114)
3) 200 μL red blood cell extract+50 μL alkaline phosphatase buffer containing 0.5 mg of uridine standard (Sigma U6381) for spike
4) 200 μL red blood cell extract+50 μL alkaline phosphatase buffer containing 200 units of alkaline phosphatase and 0.5 mg of uridine standard

Each mixture was vortexed and incubated for 30 mM at 37° C.

### High Performance Liquid Chromatography (HPLC) Analysis

Analysis by HPLC was carried out on each mixture using the following conditions:
Mobile phase: 10 mM potassium phosphate dibasic (Sigma P5833), 10 mM potassium chloride (Sigma P5405) and 10 mM Tetrabutylammonium phosphate monobasic (TBA, Sigma 268100) in 1 L of methanol/water (15/100), pH adjusted to 4.0. Column: Kinetex 2.6 u XB-C18 100 A, 150×4.6 mm (Phenomenex)
Flow rate: 0.4 mL/min
Injection volume: 5 μL or 10 μL
Detection: UV at 260 nm

### Results

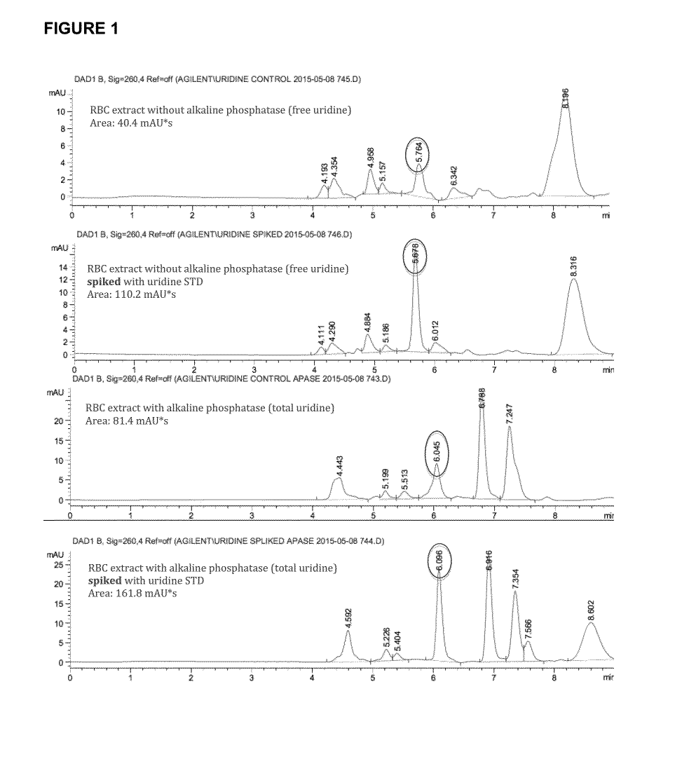

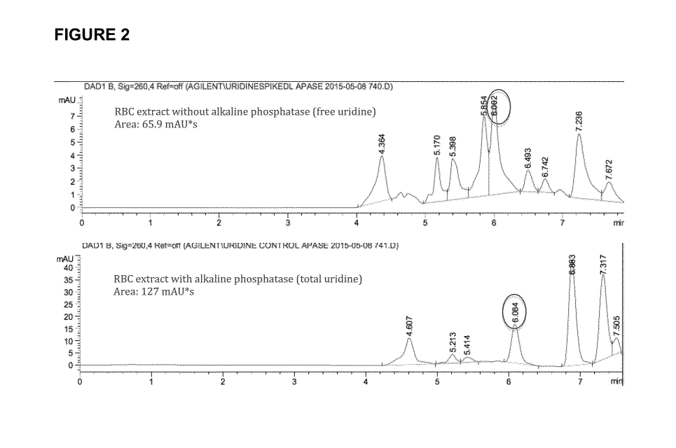

The HPLC chromatograms (see FIGS. 1 and 2) show that both free uridine and total uridine in red blood cell extracts can by detected using the above HPLC conditions. As shown in FIG. 1, the retention time of free uridine is about 5.7 min and the retention time of total uridine is about 6.0 min (5 μL injection of sample).

1. A method for treating or preventing fatigue, for promoting synthesis of adenosine triphosphate (ATP) and/or for boosting energy levels, the method comprising administering to a subject in need thereof an effective amount of two or more probiotic bacterial strains selected from Lactobacillus acidophilus, Streptococcus thermophilus, and Bifidobacterium bifidum.

2. A method according to claim 1, wherein the fatigue results from, or is associated with, stress, sleep deprivation, diet, age and/or sore muscles.

3. A method according to claim 1, wherein the method comprises the administration of a combination of Lactobacillus acidophilus, Streptococcus thermophilus, and Bifidobacterium bifidum.

4. A method according to claim 3, wherein the treatment further comprises administering to the subject an effective amount of one or more of the following: orotic acid or a salt thereof; carnitine; and coenzyme Q10.

5. A method according to claim 4, wherein the orotic acid salt is selected from magnesium orotate, zinc orotate, calcium orotate, chromium orotate, potassium orotate, copper orotate, iron orotate, manganese orotate, sodium orotate, choline orotate and lithium orotate.

6. A method according to claim 5, wherein the method comprises the administration of a combination of L. acidophilus, B. bifidum, S. thermophilus, magnesium orotate and coenzyme Q10.

7. A method for treating or preventing muscle aches, muscle pain, muscle fatigue and/or muscle degeneration or for promoting muscle repair, the method comprising administering to a subject in need thereof an effective amount of two or more probiotic bacterial strains selected from Lactobacillus acidophilus, Streptococcus thermophilus, and Bifidobacterium bifidum.

8. A method according to claim 7, wherein the muscle degeneration results from, or is associated with, age-related, injury-related muscle weakness or atrophy, or a muscular dystrophy.

9. A probiotic composition for the treatment or prevention of fatigue, for promoting synthesis of adenosine triphosphate (ATP), for boosting energy levels, for treating or preventing muscle aches, muscle pain, muscle fatigue and/or muscle degeneration, and/or for promoting muscle repair wherein the composition comprises two or more bacterial strains selected from Lactobacillus acidophilus, Streptococcus thermophilus, and Bifidobacterium bifidum.

10. A probiotic composition according to claim 9, wherein the fatigue results from, or is associated with, stress, sleep deprivation, diet, age, and/or sore muscles.

11. A probiotic composition according to claim 9, wherein the muscle degeneration results from, or is associated with, age-related, injury-related muscle weakness or atrophy, or a muscular dystrophy.

12. A probiotic composition according to claim 9, comprising L. acidophilus, B. bifidum, and S. thermophilus.

13. A probiotic composition according to claim 12, wherein the composition further comprises one or more of the following: orotic acid or a salt thereof; carnitine; and coenzyme Q10.

14. A probiotic composition according to claim 13, wherein the orotic acid salt is selected from magnesium orotate, zinc orotate, calcium orotate, chromium orotate, potassium orotate, copper orotate, iron orotate, manganese orotate, sodium orotate, choline orotate and lithium orotate.

15. A probiotic composition according to claim 14, wherein the composition comprises L. acidophilus, B. bifidum, S. thermophilus, magnesium orotate and coenzyme Q10.

16. A probiotic composition according to claim 15, wherein the composition is in a form suitable for oral administration.

17. A probiotic composition according to claim 16, in the form of capsules.
